(19) United States
(12) Patent Application Publication
(10) Pub. No.: US 2015/0366863 A1
(43) Pub. Date: Dec. 24, 2015

(54) Solid oral pharmaceutical compositions comprising fixed dose combination of metformin and sitagliptin or salts thereof

(71) Applicant: WOCKHARDT LIMITED, Aurangabad (IN)

(72) Inventors: Girish Kumar JAIN, Delhi 4 (IN); Venkataramana NAIDU, Andhra Pradesh (IN); Atul PATIL, Goa (IN)

(21) Appl. No.: 14/766,939

(22) PCT Filed: Mar. 24, 2014
(86) PCT No.: PCT/IB2014/060081

§ 371 Date: Aug. 10, 2015

(30) Foreign Application Priority Data
Mar. 28, 2013 (IN) 1222/MUM/2013
Mar. 28, 2013 (IN) 1223/MUM/2013
Mar. 28, 2013 (IN) 1224/MUM/2013
Mar. 28, 2013 (IN) 1225/MUM/2013

(51) Int. Cl. A61K 31/4985; A61K 9/24; A61K 31/155

(52) U.S. Cl. CPC A61K 31/4985; A61K 31/155; A61K 9/209

(57) ABSTRACT

The present invention provides solid oral pharmaceutical compositions comprising combination of metformin and sitagliptin or salts thereof. In particular, the present invention relates to a pharmaceutical composition comprising metformin and sitagliptin or salts thereof which is glidant and/or surface active agent. The invention also includes process of preparing such compositions and method of use of such compositions for treating type II diabetes.

### FIELD OF THE INVENTION

The present invention provides solid oral pharmaceutical compositions comprising combination of metformin and sitagliptin or salts thereof. In particular, the present invention relates to a pharmaceutical composition comprising metformin and sitagliptin or salts thereof which is devoid of glidant and/or surface active agents. The invention also includes process of preparing such compositions and method of use of such compositions for treating type II diabetes.

### BACKGROUND OF THE INVENTION

Type 2 diabetes is the most common form of diabetes and it is one of the most prevalent chronic diseases. Treatment of type 2 diabetes initially starts with diet and exercise, followed by oral antidiabetic monotherapy. During long-term treatment these regimens do not sufficiently control hyperglycemia in many patients, leading to a requirement for combination therapy within several years following diagnosis. However, co-prescription of two or more oral antidiabetic drugs may result in treatment regimens that are complex and difficult for many patients to follow. Combining two or more oral antidiabetic agents into a single tablet provides a potential means of delivering combination therapy without adding to the complexity of patients' daily regimens. Such formulations have been well accepted in other disease indications also, such as hypertension (Hyzaar®, a combination of losartan potassium and hydrochlorothiazide) and cholesterol lowering (Vytorin®, a combination of simvastatin and ezetimibe). Similarly, examples of marketed combination tablets containing two oral antidiabetic agents include Glucovance® (metformin and glyburide), and Metaglip® (metformin and glipizide).

A key step in the design of a combination tablet is selection of effective and well-tolerated treatments. Moreover, it is essential that the components have complementary mechanisms of action and compatible pharmacokinetic profiles.

Sitagliptin is an orally active inhibitor of the dipeptidyl peptidase-4 (DPP-4) enzyme. Chemically, sitagliptin is 7-[(3R)-3-amino-1-oxo-4-(2,4,5-trifluorophenyl)butyl]-5,6,7,8 tetrahydro-3-(trifluoromethyl)-1,2,4-triazolo[4,3-a]pyrazine phosphate (1:1) monohydrate with the following structure:

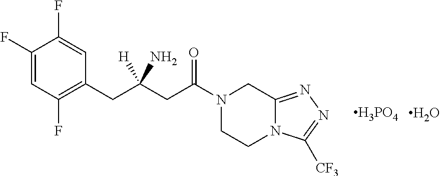

Sitagliptin phosphate is indicated as an adjunct to diet and exercise to improve glycemic control in adults with type 2 diabetes mellitus. It is a DPP-4 inhibitor, which slows down the inactivation of incretin hormones. The incretins are part of an endogenous system involved in the physiologic regulation of glucose homeostasis. When blood glucose concentrations are normal or elevated, Glucagon like peptide-1 (GLP-1) and Gastric Inhibitory Peptide (GIP) increase insulin synthesis and release from pancreatic beta cells by intracellular signaling pathways involving cyclic AMP. Sitagliptin is marketed in the United States in the form of tablets under brand name Januvia®.

Metformin is the member of the biguanide class of an oral antihyperglycemics and available in various salt forms, e.g. hydrochloride. Metformin is used in the management of type 2 diabetes mellitus. It is an antihyperglycemic agent which improves glucose tolerance in patients with type 2 diabetes, lowering both basal and postprandial plasma glucose. Chemically, metformin hydrochloride is 1-carbamimidamido-N, N-dimethylmethanimidamide hydrochloride with the following structure:

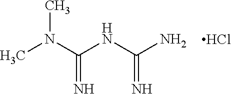

Pharmacologic mechanism of action of metformin is different from other classes of oral antihyperglycemic agents. Metformin decreases hepatic glucose production, decreases intestinal absorption of glucose, and improves insulin sensitivity by increasing peripheral glucose uptake and utilization. Unlike sulfonylureas, metformin does not produce hypoglycemia in either patients with type 2 diabetes or normal subjects, except in special circumstances) and does not cause hyperinsulinemia. Metformin is marketed in the United States in the form of extended release tablets under brand names Fortamet®, Glucophage® and Glumetza®.

A combination therapy of sitagliptin with metformin HCl (a well established active ingredient of diabetes management) provides even more effective treatment of type II diabetes. Although metformin is effective at lowering blood glucose levels, its use is associated with gastrointestinal (GI) adverse effects, particularly diarrhea and nausea. These adverse effects may limit the tolerated dose of metformin and cause patients to discontinue the therapy.

Extended-release formulations of metformin have advantages over immediate-release in terms of affording a more uniform maintenance of blood plasma active drug concentrations and providing better patient compliance by reducing the frequency of administration required.

Numerous studies have been conducted to address the formulation and drug release systems of combination of antidiabetic drugs and attempts have been made to achieve the combination formulation having desired dissolution profile.

U.S. Pat. No. 6,340,475 discloses a controlled-release oral drug dosage formulation designed for gastric retention and controlled delivery of metformin into the gastric cavity.

Extended-release formulations of metformin are disclosed in several other U.S. Pat. Nos. 6,635,280; 6,866,866; 6,475,521 and 6,660,300.

European patent application No. EP 1537880 A1 discloses a sustained release formulation of DPP-IV inhibitors and a hydrophilic polymer.

U.S. Application publication No. US 20070172525 and US 20080064701 discloses pharmaceutical composition comprising a DPP-4 inhibitor and a slow-release form of metformin.

PCT publication No. WO 2009111200 discloses a formulation comprising an inner core comprising metformin hydrochloride. The inner core is coated with a sustained-release polymer and further comprises a coating comprising an immediate release composition of sitagliptin.

PCT publication number WO 2009099734 discloses pharmaceutical composition comprising a tablet core comprised of metformin and an extended release excipient (HPMC). The tablet core is then coated with immediate release polymer comprising sitagliptin.

U.S. Application publication No. 20090105265 discloses a fixed-dose combination of metformin and sitagliptin. The application discloses use of surfactants in the composition in order to achieve desired dissolution profile.

U.S. Application publication No. 20100330177 discloses a fixed-dose combination of metformin and sitagliptin. The application discloses use of glidant as excipient.

Various types of formulations have been suggested in the art for fixed dose combination of metformin and sitagliptin composition. The art in the broad sense teaches to incorporate glidant and/or surface active agents in the formulation in order to achieve the consistent tablet weight, dose uniformity and desired dissolution profile.

There is still exists an enduring need for an alternate, improved and stable fixed dose combination formulation of metformin and sitagliptin.

### SUMMARY OF THE INVENTION

In one general aspect, there is provided a solid oral pharmaceutical composition comprising sitagliptin or a salt thereof, metformin or a salt thereof and one or more pharmaceutically acceptable excipients, wherein the composition is devoid of glidant and/or surface active agents.

In another general aspect, there is provided a solid oral pharmaceutical composition comprising about 3 to 20% by weight of sitagliptin or a salt thereof; about 25 to 94% by weight of metformin or a salt thereof; about 0.1 to 10% by weight of a lubricant, and about 0 to 35% by weight of a binding agent.

In another general aspect, there is provided a solid oral pharmaceutical composition comprising at least one component of sitagliptin or a salt thereof, at least one component of metformin or a salt thereof and one or more pharmaceutically acceptable excipients, wherein the composition is devoid of glidant and/or surface active agents.

In another general aspect, there is provided a solid oral pharmaceutical composition comprising sitagliptin or a salt thereof, metformin or a salt thereof and one or more pharmaceutically acceptable excipients, wherein the pharmaceutically acceptable excipients comprises one or more diluents, disintegrants, binder, anti-oxidants; and the composition is devoid of glidant and/or surface active agent.

In another general aspect, the solid oral pharmaceutical composition is in the form of a multilayer tablet, a bilayer tablet or a trilayer tablet.

In another general aspect, there is provided a solid oral pharmaceutical composition comprising sitagliptin or a salt thereof, metformin or a salt thereof and one or more pharmaceutically acceptable excipients, wherein the composition retains at least 90% w/w of the total potency of metformin and sitagliptin or salts thereof after storage at 30° C. and 60% relative humidity for at least 3 months and characterized in that the composition is devoid of glidant.

In another general aspect, there is provides a solid oral pharmaceutical composition of metformin or salts thereof and sitagliptin or salts thereof prepared by dry granulation, wet granulation, slugging or direct compression.

In another general aspect, there is provided a solid oral pharmaceutical composition comprising at least one immediate release component comprising sitagliptin or a salt thereof, at least one extended release component comprising metformin or a salt thereof, and one or more pharmaceutically acceptable excipients; wherein the composition is devoid of glidant and/or surface active agents.

In another general aspect, the extended release component of the solid oral pharmaceutical composition constitute either a matrix of metformin or salts thereof, one or more pharmaceutical excipients and one or more rate controlling agents, or a compressed layer of metformin or salts thereof and one or more pharmaceutical excipients coated with one or more rate controlling agents, or both.

In another general aspect, there is provided a solid oral pharmaceutical composition comprising:
(a) at least one first component comprising sitagliptin, metformin or salts thereof and one or more pharmaceutical excipients exhibiting immediate release;
(b) at least one second component comprising metformin or salt thereof and one or more pharmaceutical excipients exhibiting extended release, and
(c) at least one third component comprising metformin or salts thereof and one or more pharmaceutical excipients exhibiting immediate release, wherein the first and second components are at last partially coated with third component, and characterized in that the composition is devoid of glidant and/or surface active agents.

In another general aspect, there is provided a process for preparing a solid oral pharmaceutical composition comprising sitagliptin or a salt thereof and metformin or a salt thereof, the process comprises the steps of:
(a) providing at least one component comprising metformin or a salt thereof, optionally with sitagliptin or a salt thereof;
(b) providing at least one component comprising sitagliptin or a salt thereof, optionally with metformin or a salt thereof; and
(c) combining the components of step (a) and (b),
wherein the composition is devoid of glidant and/or surface active agents.

In another general aspect, there is provided a method of treating Type 2 diabetes in a patient which method comprises administering the solid oral pharmaceutical composition as substantially described herein.

### DETAILED DESCRIPTION OF THE INVENTION

The inventors of the present invention have surprisingly found that it is possible to achieve metformin and sitagliptin combination composition having desired dissolution profile without using glidant and/or surface active agents, the resulting formulation may also posses enhanced formulation robustness and stability.

In particular, the inventors have found that when the composition of metformin and sitagliptin is devised by judicially using pharmaceutical excipients other than glidants and/or surface active agents, the resulting formulation may exhibit bioequivalency to equivalent doses of their single entity formulations and or achieve consistent tablet weight and dose uniformity.

The present invention relates to solid oral pharmaceutical composition comprising sitagliptin or a salt thereof, metformin or a salt thereof and one or more pharmaceutically acceptable excipients, wherein the composition is devoid of glidant and/or surface active agents.

The term “compartment” used herein throughout the specification is used to intend a part of the dosage form comprising one or both of metformin and sitagliptin, and optional other active ingredients, optionally together with pharmaceutical excipients. The part of the dosage form can be in the form of a layer (formed by coating or compression), granules, pellets, tablets, or mini-tablets. Preferably, the compartments comprise a homogenous mixture of components. In each compartment, at least one type of active ingredient is contained.

In one embodiment, at least in one, optionally in two compartments both metformin and sitagliptin are present. The compartments can comprise immediate or extended release compositions. According to the invention, the composition is devoid of glidant and/or surface active agents.

Preferably, the compartments are provided in the form of a layer. The pharmaceutical dosage form comprising the compartments will then represent a bilayer tablet, a trilayer tablet or a multilayer tablet, preferably bilayer tablet.

The term “tablet” used throughout the specification refers to and intended to encompass compressed pharmaceutical dosage formulations of all shapes and sizes, whether coated or uncoated.

The term “layer” used throughout the specification refers to denote a spatial part of the pharmaceutical composition or dosage form other than that formed by applying a coating.

The term “coating” used throughout the specification refers to a layer which at least partly covers an object and is applied by various coating processes known in the art.

The terms “metformin” and “sitagliptin” used throughout the specifications refers to any pharmaceutically acceptable salts of metformin and sitagliptin. The preferred salt of metformin is metformin hydrochloride. The preferred salt of sitagliptin is sitagliptin phosphate, more preferably its monohydrate.

The term “immediate release” used throughout the specification refers that within 2 hours, preferably within 1.5 hour, more preferably within 1 hour and most preferably within 30 minutes, at least 80%, preferably at least 85%, more preferably at least 90% of the drug being present in the compartment is dissolved or released.

The term “extended release” used throughout the specification refers that at least 95% of the drug being present in the component is not dissolved or released, not before 2 hours, preferably not before 3 hours, and more preferably not before 4 hours.

A suitable test for determining the dissolution is the test using Apparatus 2 according to the US Pharmacopoeia 32-NF 27, described in General chapter 711 (Dissolution). Conditions chosen for the test were Apparatus 2 with 100 rpm in phosphate buffer medium pH 6.8.

In another embodiment, the solid oral pharmaceutical composition is in the form of a multilayer tablet, a bilayer tablet or a trilayer tablet.

In an embodiment, the first and second compartments employed in the composition of the invention may include polymers and pharmaceutically acceptable excipients to enable formation of a bilayer coated tablet.

In another embodiment, the extended release compartment in the composition of the present invention may contain additional anti-diabetic agents other than metformin.

The inventors of the present invention have further determined that if the composition of the present invention is formulated without using glidant, particularly in the core of the composition, the composition may achieve consistent tablet weight and dose uniformity.

In another embodiment, the extended release compartment of the composition is substantially free of surface active agents.

In another embodiment, the solid oral pharmaceutical composition of the invention is substantially free of surface active agents.

In another embodiment, the extended release compartment according to present invention does not contain disintegrants and wherein the immediate release compartment contains one or more disintegrants but no rate controlling agent.

In another embodiment, the first and third compartment according to the present invention does not comprise any rate controlling agent, in particular not the rate controlling agent that used in the first compartment.

Suitable rate controlling agents may be selected from the group consisting of hydrophilic agents (e.g. water-soluble polymers), lipophilic agents (water-insoluble polymers) and inert matrix agents, wherein the hydrophilic agents are selected from the group of pharmaceutical excipients which generate a gel in contact with water, including cellulose derivatives such as hydroxypropyl methyl cellulose, hydroxyethyl cellulose, hydroxypropyl cellulose, methyl cellulose and the like; noncellulose polysaccharides such as galactomannanes, guar gum, carob gum, gum arabicum, alginates, pectins, and the like; polyvinylpyrrolidone; polyvinylacetate polymers and copolymers; acrylic acid polymers and copolymers, polyethylene oxide and mixtures thereof; the lipophilic agents are selected from the group consisting of waxes such as white wax, bees wax, carnauba wax and the like; fatty acids and alcohols such as stearic acid, palmitic acid, lauric acid and the like, and cetyl alcohol, cetostearyl alcohol, stearyl alcohol and the like; fatty acids esters such as monostearates of propylene glycol and fatty acid esters of sucrose, sucrose distearate and the like; and glycerides such as mono-, di- or triglycerides, e.g. palmitin, stearin, behenic, laurin, myristin, hydrogenated vegetable, castor, cottonseed oils, glyceril behenate and the like; ethyl cellulose; acrylic acid polymers and copolymers (available commercially under Eudragit® brand); and mixtures thereof; and the inert agents are selected from the group consisting of thermoplastic polymers, which are insoluble and indigestible in the gastrointestinal fluids, such as polyvinyl chloride, polyethylene, vinyl acetate/vinyl chloride copolymers, polymethylmethacrylates, polyamides, silicones, ethyl cellulose, polystyrene, and mixtures thereof. The amount of rate controlling agent in the composition ranges from about 10 to about 50% w/w, preferably from about 15% to about 45% by weight of the composition.

The oral solid dosage form composition of the present invention further comprises various pharmaceutical excipients suitable for oral administration. Such excipients are selected from the group consisting of binding agents, fillers, filler-binders, disintegrants, lubricants, sweeteners, flavourings and colouring agents, preferably the excipients are selected from the group consisting of binding agents, filler-binders, and lubricants.

The fillers and/or filler-binder are selected from the group consisting of different grades of starches, such as maize starch, potato starch, rice starch, wheat starch, pregelatinized starch, fully pregelatinized starch, cellulose, such as microcrystalline cellulose or silicified microcrystalline cellulose, mannitol, erythritol, lactose, such as lactose monohydrate and lactose anhydrous, calcium salts, such as calcium hydrogen phosphate dihydrate, anhydrous dibasic calcium phosphate, sorbitol, and xylitol, particularly preferred, the fillers and/or filler-binders are selected from the group consisting of pregelatinized starch, microcrystalline cellulose, lactose monohydrate, and lactose, even further preferred the filler and/or filler-binder is selected from the group consisting of microcrystalline cellulose and anhydrous dibasic calcium phosphate.

The lubricants are selected from the group consisting of stearic acid, sodium stearyl fumarate and magnesium stearate, particularly preferred, the lubricant is magnesium stearate.

Binding agents are selected from the group consisting of polyvinyl pyrrolidone (Povidone), copolymers of vinylpyrrolidone with other vinylderivatives (Copovidone), hydroxypropyl methylcellulose, methylcellulose, hydroxypropylcellulose, powdered acacia, gelatin, guar gum, carbomer such as carbopol, polymethacrylates and starch.

In an embodiment, the immediate release (second and/or third) compartment additionally comprises disintegrants.

The solid oral pharmaceutical composition of the present invention can be prepared by methods known to the person skilled in the art.

Preferably, the components comprising metformin or salts thereof and sitagliptin or salts thereof are formed by dry granulation, wet granulation, slugging or direct compression or by coating process. The compartments then can be processed in different orders and methods known to the person skilled in the art to form a dosage form.

In an embodiment, the process of preparing the solid oral pharmaceutical composition of metformin and sitagliptin or salts thereof comprises steps of:
(a) providing at least one component comprising metformin or a salt thereof, optionally with sitagliptin or a salt thereof;
(b) providing at least one component comprising sitagliptin or a salt thereof, optionally with metformin or a salt thereof; and
(c) combining the components of step (a) and (b),
wherein the composition is devoid of glidant and/or surface active agents.

In a further embodiment, granulation liquids can be added, especially in second compartment, if the composition comprises metformin or pharmaceutically acceptable salts thereof, as also described elsewhere herein. Granulation liquid is removed during further processing of the respective compositions, however, some residual water is required in order to render granulate compressible.

In another embodiment, the solid oral composition is in the form of a bilayer tablet and comprises a first layer comprising 90% of metformin or salts thereof exhibiting extended release, a second layer comprising sitagliptin or salts thereof and 5% metformin or salt thereof exhibiting immediate release and an immediate-release coating over the two layers comprising 5% of metformin or salts thereof. In a further preferred embodiment, the first layer is devoid of glidant. In a further preferred embodiment, the tablet is devoid of surface active agents.

The present invention is further illustrated by the following examples which are provided merely to be exemplary of the invention and do not limit the scope of the invention. Certain modifications and equivalents will be apparent to those skilled in the art and are intended to be included within the scope of the present invention.

### Example 1

### Sitagliptin Phosphate and Metformin Extended Release Tablet

TABLE 1

| | | |
|---|---|---|
| Sr. | | Qty per Tablet |
| No. | Ingredients | (% w/w) |

First component (Extended Release Granules)

| | | |
|---|---|---|
| 1 | Metformin HCl | 10-40 |
| 2 | Microcrystalline Cellulose | 5-50 |
| 3 | Maize Starch | 5-30 |
| 4 | Hypromellose 2208 | 10-40 |
| 5 | Carbopol | 5-30 |
| 6 | Water | q.s. |
| 7 | Magnesium stearate | 1-3 |

Second component (Immediate Release Granules)

| | | |
|---|---|---|
| 8 | Metformin HCl | 1-10 |
| 9 | Sitagliptin Phosphate | 10-50 |
| 10 | PVP | 10-70 |
| 11 | Kollidon VA 64 | 10-70 |
| 12 | Water | q.s. |
| 13 | Magnesium stearate | 1-3 |

Third Component (Coating)

| | | |
|---|---|---|
| 14 | Metformin HCl | 1-10 |
| 15 | Opadry White | 20-60 |
| 16 | PEG 4000 | 10-70 |
| 17 | Water | q.s. |

Process: First (Extended Release Granules) component of Metformin HCl was prepared by mixing Metformin, Microcrystalline cellulose, Maize starch, Hypromellose 2208, Carbopol with water. The mixture was granulated to form granules. The granules were then lubricated with Magnesium stearate. The second component (Immediate Release Granules) component of Metformin HCl and Sitagliptin Phosphate was prepared by mixing Metformin, Sitagliptin, PVP, Kollidon VA 64 with water. The mixture was granulated to form granules. The granules were then lubricated with Magnesium stearate.

The first (Extended Release Granules) and second components (Immediate Release Granules) were then compressed to form a tablet.

The tablet was then further coated with a mixture of Metformin HCl, Opadry white, PEG 4000 and water.

### Example 2

### Sitagliptin Phosphate and Metformin Extended Release Tablet

TABLE 2

| | | |
|---|---|---|
| Sr. | | Qty per Tablet |
| No. | Ingredients | (% w/w) |

First component (Extended Release Granules)

| | | |
|---|---|---|
| 1 | Metformin HCl | 10-40 |
| 2 | Microcrystalline Cellulose | 5-50 |
| 3 | Maize Starch | 5-30 |
| 4 | Hypromellose 2208 | 10-40 |
| 5 | Carbopol | 5-30 |
| 6 | Water | q.s. |
| 7 | Talc | 1-3 |

Second component (Immediate Release Granules)

| | | |
|---|---|---|
| 8 | Metformin HCl | 1-10 |
| 9 | Sitagliptin Phosphate | 10-50 |
| 10 | PVP | 10-70 |
| 11 | Kollidon VA 64 | 10-70 |
| 12 | Water | q.s. |
| 13 | Magnesium stearate | 1-3 |

Third Component (Coating)

| | | |
|---|---|---|
| 14 | Metformin HCl | 1-10 |
| 15 | Opadry White | 20-60 |
| 16 | PEG 4000 | 10-70 |
| 17 | Water | q.s. |

Process: First (Extended Release Granules) component of Metformin HCl was prepared by mixing Metformin, Microcrystalline cellulose, Maize starch, Hypromellose 2208, Carbopol with water. The mixture was granulated to form granules. The granules were then lubricated with talc.

The second component (Immediate Release Granules) component of Metformin HCl and Sitagliptin Phosphate was prepared by mixing Metformin, Sitagliptin, PVP, Kollidon VA 64 with water. The mixture was granulated to form granules. The granules were then lubricated with Magnesium stearate.

The first (Extended Release Granules) and second components (Immediate Release Granules) were then compressed to form a tablet.

The tablet was then further coated with a mixture of Metformin HCl, Opadry white, PEG 4000 and water.

### Example 3

### Sitagliptin Phosphate and Metformin Extended Release Tablet

TABLE 3

| | | |
|---|---|---|
| Sr. | | Qty per Tablet |
| No. | Ingredients | (% w/w) |

First component (Extended Release Granules)

| | | |
|---|---|---|
| 1 | Metformin HCl | 10-40 |
| 2 | Microcrystalline Cellulose | 5-50 |
| 3 | Maize Starch | 5-30 |
| 4 | Hypromellose 2208 | 10-40 |
| 5 | Sodium Lauryl Sulfate | 0.1-3 |
| 6 | Carbopol | 5-30 |
| 7 | Water | q.s. |
| 8 | Magnesium stearate | 1-3 |

Second component (Immediate Release Granules)

| | | |
|---|---|---|
| 9 | Metformin HCl | 1-10 |
| 10 | Sitagliptin Phosphate | 10-50 |
| 11 | PVP | 10-70 |
| 12 | Kollidon VA 64 | 10-70 |
| 13 | Water | q.s. |
| 14 | Magnesium stearate | 1-3 |

Third Component (Coating)

| | | |
|---|---|---|
| 15 | Metformin HCl | 1-10 |
| 16 | Opadry White | 20-60 |
| 17 | PEG 4000 | 10-70 |
| 18 | Water | q.s. |

Process: First (Extended Release Granules) component of Metformin HCl was prepared by mixing Metformin, Microcrystalline cellulose, Maize starch, Hypromellose 2208, Sodium Lauryl Sulfate and Carbopol with water. The mixture was granulated to form granules. The granules were then lubricated with Magnesium stearate.

The second component (Immediate Release Granules) component of Metformin HCl and Sitagliptin Phosphate was prepared by mixing Metformin, Sitagliptin, PVP, Kollidon VA 64 with water. The mixture was granulated to form granules. The granules were then lubricated with Magnesium stearate.

The first (Extended Release Granules) and second components (Immediate Release Granules) were then compressed to form a tablet.

The tablet was then further coated with a mixture of Metformin HCl, Opadry white, PEG 4000 and water.

1. A solid oral pharmaceutical composition comprising sitagliptin or a salt thereof, metformin or a salt thereof and one or more pharmaceutically acceptable excipients, wherein the composition is devoid of glidant and/or surface active agent.

2. The solid oral pharmaceutical composition of claim 1, wherein the composition comprising about 3 to 20% by weight of sitagliptin or a salt thereof; about 25 to 94% by weight of metformin or a salt thereof; about 0.1 to 10% by weight of a lubricant; and about 0 to 35% by weight of a binding agent.

3. The solid oral pharmaceutical composition of claim 1, wherein the composition comprises at least one immediate release component comprising sitagliptin or a salt thereof, at least one extended release component comprising metformin or a salt thereof.

4. The solid oral pharmaceutical composition of claim 3, wherein the extended release component in the composition constitute a matrix of metformin or salts thereof, one or more pharmaceutical excipients and one or more rate controlling agents.

5. The solid oral pharmaceutical composition of claim 3, wherein the extended release component in the composition constitute a compressed layer of metformin or salts thereof and one or more pharmaceutical excipients coated with one or more rate controlling agents, or both.

6. The solid oral pharmaceutical composition of claim 4 or 5, wherein the rate controlling agent comprises one or more hydrophilic agents, lipophilic agents and inert matrix agents or mixtures thereof.

7. A process for preparing the solid oral pharmaceutical composition of claim 5, which process comprises the steps of:
(a) providing at least one component comprising metformin or a salt thereof, optionally with sitagliptin or a salt thereof;
(b) providing at least one component comprising sitagliptin or a salt thereof, optionally with metformin or a salt thereof, and
(c) combining the components of step (a) and (b).

8. A multilayer tablet comprising:
(a) at least one layer comprising sitagliptin or a salt thereof, optionally metformin or a salt thereof and one or more pharmaceutical excipients exhibiting immediate release, and
(b) at least one layer comprising metformin or salt thereof and one or more pharmaceutical excipients exhibiting extended release,
wherein the composition is devoid of glidant and/or surface active agent.

9. A solid oral pharmaceutical composition comprising:
(a) at least one first component comprising sitagliptin, metformin or salts thereof and one or more pharmaceutical excipients exhibiting immediate release;
(b) at least one second component comprising metformin or salt thereof and one or more pharmaceutical excipients exhibiting extended release, and
(c) at least one third component comprising metformin or salts thereof and one or more pharmaceutical excipients exhibiting immediate release, wherein the first and second components are at last partially coated with third component; and
characterized in that the composition is devoid of glidant and/or surface active agents.

10. The solid oral pharmaceutical composition of claim 1 or 9, wherein the composition is in the form of a bilayer tablet, a trilayer tablet or a multilayer tablet.

11. Use of the solid oral pharmaceutical composition of claim 1 or 9 for treating Type 2 diabetes in a patient.
